(19) United States
(12) Patent Application Publication
(10) Pub. No.: US 2004/0029262 A1
(43) Pub. Date: Feb. 12, 2004

(54) Organic material treatment apparatus, system and method

(76) Inventor: Robert John Walker, Helena Valley (AU)

Correspondence Address: Gregory P LaPointe, Bachman & LaPointe, Suite 1201, 900 Chapel Street, New Haven, CT, 06510, US

(21) Appl. No.: 10/363,893

(22) PCT Filed: Sep. 10, 2001
(86) PCT No.: PCT/AU01/01140

(30) Foreign Application Priority Data
Sep. 8, 2000 (AU) PR 0019
Nov. 15, 2000 (AU) PR 1500
Apr. 17, 2001 (AU) PR 4428

(51) Int. Cl. C12M 1/00

(52) U.S. Cl. 435/290.1

(57) ABSTRACT

The invention relates to a container, a system and an apparatus for the treatment of organic matter involving the use of worms to decompose organic material and produce castings. The container includes an inner housing, housing having walls with apertures, an in-feeding means, a collection means and a discharge means. The system is a plurality of such containers having a common organic matter delivery means and a common discharge means. The apparatus comprises a pair of wheel assemblies which can be mounted to opposing ends of a container, a framework for supporting the wheel assemblies and a drive means to rotate the container.

### FIELD OF THE INVENTION

This invention relates to the treatment of organic matter to cause decomposition and to the treatment of the decomposed matter using worms.

Throughout the specification, unless the context requires otherwise, the word “comprise” or variations such as “comprises” or “comprising”, will be understood to imply the inclusion of a stated integer or group of integers but not the exclusion of any other integer or group of integers.

### BACKGROUND ART

In my Provisional Patent Applications Nos PR0019 dated Sep. 8, 2000 and PRI500 dated Nov. 15, 2000, I describe apparatus, systems and methods for decomposing organic materials and systems and methods for the treatment of organic materials using worms. The merits of decomposing organic matter to form compost are well known as are the merits of using worms to produce worm castings. Such merits are described in greater detail in the provisional specifications associated with the above Provisional Patent Applications, and the contents of each provisional specification is incorporated in the present specification by cross reference.

### DISCLOSURE OF THE INVENTION

The present applicant has determined that the worm casting production process is accelerated or improved by using the decomposed organic material produced in accordance with the invention of Provisional PR1500 as feed material for the worm container and process described in Provisional PR0019.

The invention therefore provides, in a first aspect, a process for producing useful organic material including the step of feeding at least partly decomposed organic material to a container and introducing worms into the container to further decompose said organic material to produce castings, and collecting said castings.

In one form, the container includes:

an inner housing for accommodating organic matter and worms within the confines of the container;

an in-feeding means for feeding organic matter from outside of said container at one end, to inside and along the container, so as to fill and maintain the housing with a supply of organic matter for treatment by the worms;

the housing having walls formed with apertures sufficiently small to retain the organic matter within the housing and sufficiently large to allow for the passage of worm castings and liquid expelled from said organic matter therethrough;

collection means for collecting said castings and liquid disposed beneath the bottom of said housing, within the container; and

discharge means to selectively discharge said castings and liquid from the container.

Preferably, a closure is disposed at the other end of the container for allowing access to said housing and said collection means.

Preferably, the in-feeding means is assisted with electronic micro-switch and computerised technology to keep the container at optimal levels of capacity.

Preferably, the container includes lighting means to illuminate the outer surface of organic matter reposed within said housing. Light repels worms from the outer surface to avoid escape from the compostible material.

Preferably, a plurality of containers are arranged into a system including:

a common organic matter delivery means linking all of the containers for feeding the in-feeding means of each container with organic matter; and

a common discharge means linking all of the containers for discharging castings and/or liquid from the discharge means of each container.

Preferably, the decomposed organic matter is introduced into:

an array of rectangular, stackable containers arranged into one or more regular vertical columns and one or more horizontal rows;

each container being of a modular form and arranged in the array so that the one end of each container is disposed at a common end of the array;

the delivery means comprising a main organic matter feed means for supplying organic matter from a main supply area externally of said array to said common end and to said in-feeding means of each said container; and

the common discharge means comprising a conveyor for conveying castings and/or liquid from said discharge means of each said container from said common end to a main discharge area externally of said array.

Preferably, said containers are arranged in said array so that the longitudinal axis of each container is disposed in parallel spaced relation to each other container, and the one ends of the containers are disposed in rectilineal alignment to define said common end.

In another aspect of the present invention, there is provided an apparatus for decomposing organic matter involving the use of a replaceable container module comprising:

a pair of wheel assemblies for detachably mounting to opposing ends of a container module to form an integral unit when fixedly connected thereto;

a framework for supporting the wheel assemblies in an elevated position when the container module is supported therebetween as an integral unit for rotation about a central axis; and

drive means associated with at least one wheel assembly to drive rotation of said one wheel assembly relative to the framework about the central axis, whereby the interconnectivity of the wheel assemblies to the container module permits the container module to be rotated as a whole.

The apparatus may be used to produce decomposed or partly decomposed organic matter that may be stockpiled for subequent use elsewhere, or alternatively be used as a source of organic matter for the container as defined in the preceding aspect of the invention.

Preferably, each wheel assembly has:

(i) an inner accessway generally commensurate in shape to circumscribe and accommodate the end of a container module axially therein;

(ii) fixing means disposed within said accessway to releasably attach said wheel assembly thereto; and

(iii) an outer rim, rotatable about a central axis of the wheel assembly.

Preferably, the framework comprises a pair of end support members for rotatably supporting the respective wheel assemblies in an upright position.

Preferably, each support member has a pair of transversely spaced sheaved rollers to accommodate the rim of a corresponding wheel assembly therein in coplanar relationship therewith so that the wheel assembly reposes at the elevated position and is able to rotate about the central axis at this position in conjunction with the rollers.

Preferably, the container module is rectangular in cross-section and is provided with an opening and a sealable closure along one side thereof for filling and emptying of the contents thereof.

Preferably, the drive means comprises a reciprocating clamp and release mechanism pivotally mounted to said framework and disposed tangentially to said wheel assembly in clamping and release engagement therewith, whereby said reciprocating mechanism includes control means to clampingly engage said wheel assembly during the extension stroke of said reciprocating mechanism, thereby imparting rotation to said wheel assembly from a reference position relative to said framework, and release said wheel assembly during the retraction stroke of said reciprocating mechanism to return said reciprocating mechanism to said reference position.

Preferably, the drive means also comprises a fixed clamp and release mechanism fixedly mounted to said framework and disposed tangentially to said wheel assembly in clamping and release engagement therewith to work in co-operation with said reciprocating clamp and release mechanism, whereby said fixed mechanism includes control means to clampingly engage said wheel assembly during the retraction stroke of said reciprocating mechanism, thereby locking movement of said wheel assembly during said retraction stroke, and release said wheel assembly during the extension stroke of said reciprocating mechanism to allow rotation of said wheel assembly from said reference position.

Alternatively, the drive means may also comprise a pair of said reciprocating clamp and release mechanisms are mounted to said framework and wheel assembly and are controlled to operate in cooperation but out of phase with each other, so that whilst one reciprocating mechanism is clampingly engaged to said wheel assembly during said extension stroke thereof, the other reciprocating mechanism is released during said retraction stroke thereof, and vice versa.

Preferably, the container module comprises a standard shipping container.

Preferably, the side walls and the top of the shipping container are externally reinforced by a plurality of reinforcing members configured in a truss arrangement fixedly juxtaposed thereto.

Preferably, the framework is provided with a longitudinally disposed upright kick-board extending axially between the opposing ends of the framework to define a space either side thereof at the base of the apparatus for emptying the contents of the container module and facilitating removal therefrom

Alternative forms of drive means to that described above may be used with equally acceptable results. In one such alternative, a chain and sprocket drive is used to rotate one or both wheel assemblies directly from a motor.

In another alternative, intermeshing gear wheels are used in place of the chain and sprocket drive.

The apparatus may comprise a pair of wheel assemblies for detachably mounting to opposing ends of a container module to form an integral unit when fixedly connected thereto;

a framework for supporting the wheel assemblies in an elevated position when the container module is supported therebetween as an integral unit for rotation about a central axis; and

drive means associated with at least one wheel assembly to drive rotation of said one wheel assembly relative to the framework about the central axis, whereby the interconnectivity of the wheel assemblies to the container module permits the container module to be rotated as a whole;

wherein the unit can be rotated to a filling position at which the container module can be filled with material containing organic residues and to an emptying position at which the container module can be discharged of its contents.

In accordance with another aspect of the invention, there is provided a system for handling the disposal of large volumes of waste containing organic matter comprising:

a plurality of apparatuses of the type defined in the preceding aspect of the invention, the plurality being arranged in an array so that two or more apparatus in the array are disposed adjacent to one another to share a common filling or discharge facility.

Preferably, the common filling facility is a ramp and elevated platform disposed adjacent to a plurality of said apparatuses disposed serially in end-to-end relationship to each other, the edge of the platform being disposed marginally adjacent to the sides of the container modules at a level generally horizontal with the opening of a container module when the container module is disposed with the closure in an open position and the opening is at its uppermost juxtaposition to the platform edge.

Preferably, the common discharge facility comprises a wide belt conveyor disposed beneath the lowermost point of the container modules, extending longitudinally in parallel relationship to the central axis of each apparatus.

The decomposed organic material produced by the apparatus, system and method defined above may be transferred to the worm container by any suitable means, including direct discharge from the apparatus into the worm-containing container.

### BRIEF DESCRIPTION OF THE DRAWINGS

The invention will be better understood in the light of the following description of two specific embodiments thereof. The description is made with reference to the accompanying drawings, wherein:

FIG. 1 is a perspective view of the apparatus in accordance with the first embodiment showing the hydraulic door arrangement;

FIG. 2 is a cross-sectional end elevation of the apparatus of FIG. 1 showing the container lining;

FIG. 3 is a similar view to FIG. 1, but showing the hydraulic coupling arrangement;

FIG. 4 is a similar view to FIG. 1, but showing the aeration arrangement;

FIG. 5 is a similar view to FIG. 1, but showing the moisturising arrangement;

FIG. 6 is a similar view to FIG. 1, but showing the sensing arrangement;

FIG. 7 is a similar view to FIG. 2, but showing he retraction stroke with the container in one position;

FIG. 8 is a similar view to FIG. 7, but showing the reciprocating mechanism in the extension stroke with the container rotated slightly;

FIG. 9 is a similar view to FIG. 1, but showing the container disposed on a gradient;

FIG. 10 is a similar view to FIG. 1, but showing the container arrangement with paddles;

FIG. 11 is a conceptual oblique view of the apparatus showing a loading conveyor and discharge conveyor arrangement particular suited to the opening and door closure arrangement of the first embodiment;

FIG. 12a is a cross-sectional view of the apparatus in accordance with another embodiment provided with a container module fitted with an alternative opening and door closure arrangement rotated to a filling position;

FIG. 12b is a similar cross-sectional view as of FIG. 12a but showing the container rotated to an emptying position for removing composed waste therefrom;

FIG. 13a is a schematic end view of the large scale system for treating waste in accordance with the second embodiment;

FIG. 13b is a side sectional view of FIG. 13a.

FIG. 14 is a detailed cross sectional view of a container for treating waste organic matter in accordance with the first embodiment of the invention;

FIG. 15 is a schematic perspective view of the container of FIG. 14;

FIG. 16 is a schematic perspective view showing an array of the containers shown in FIGS. 14 and 15 arranged in a manner to provide a large scale plant for the treatment of large volumes of organic matter;

FIG. 17a is a fragmentary side elevation of the corridor between the array of containers of FIG. 16 showing the arrangement of the main delivery auger, the feed chutes and the feed hoppers relative to the containers;

FIG. 17b is a similar view to FIG. 17a, but shows the arrangement of the discharge chutes and main discharge conveyor relative to the containers;

FIG. 18 is an exploded perspective view showing the sub-frame and housing arrangement separated from the container; and

FIG. 19 is a perspective view of the system in accordance with the third embodiment.

### BEST MODE(S) FOR CARRYING OUT THE INVENTION

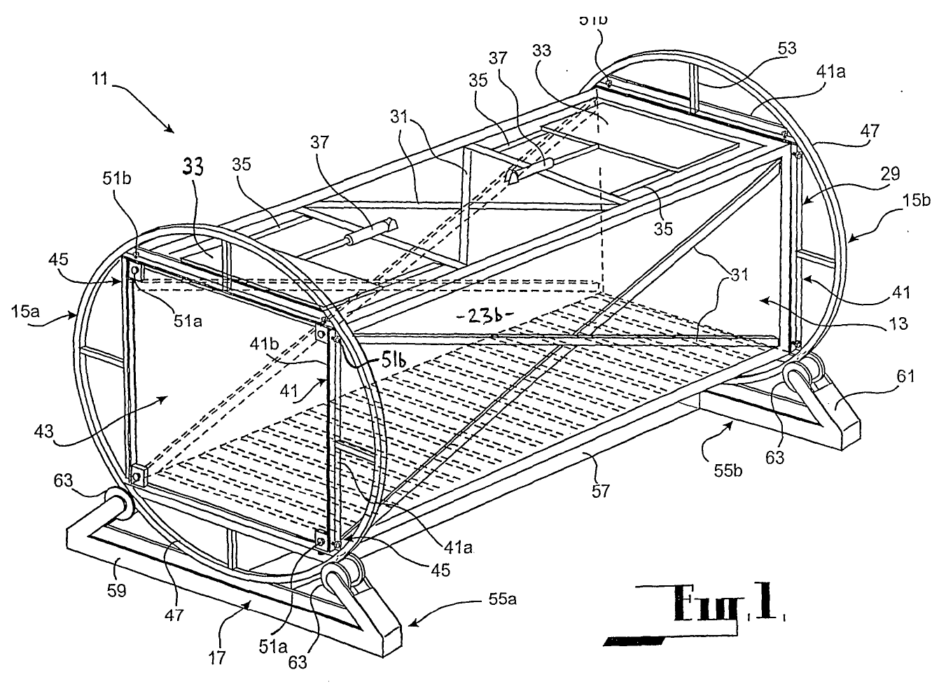

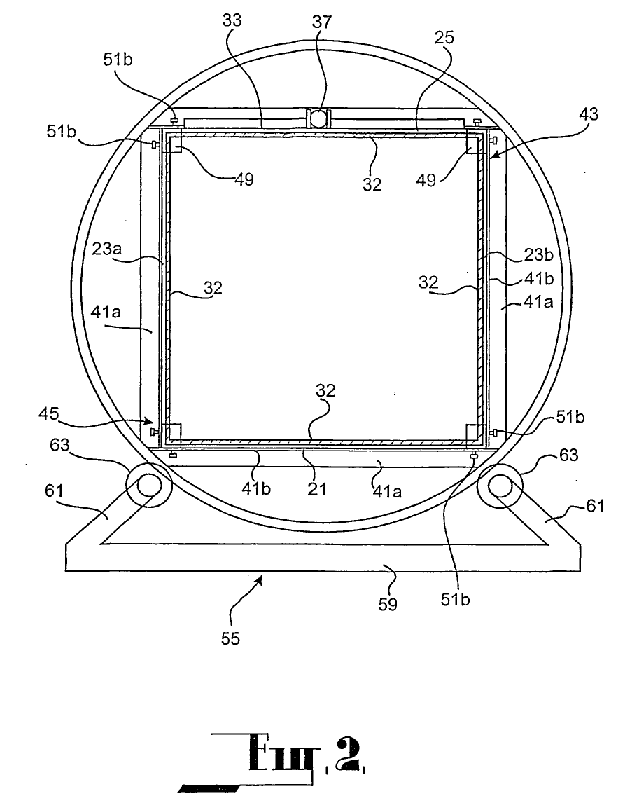

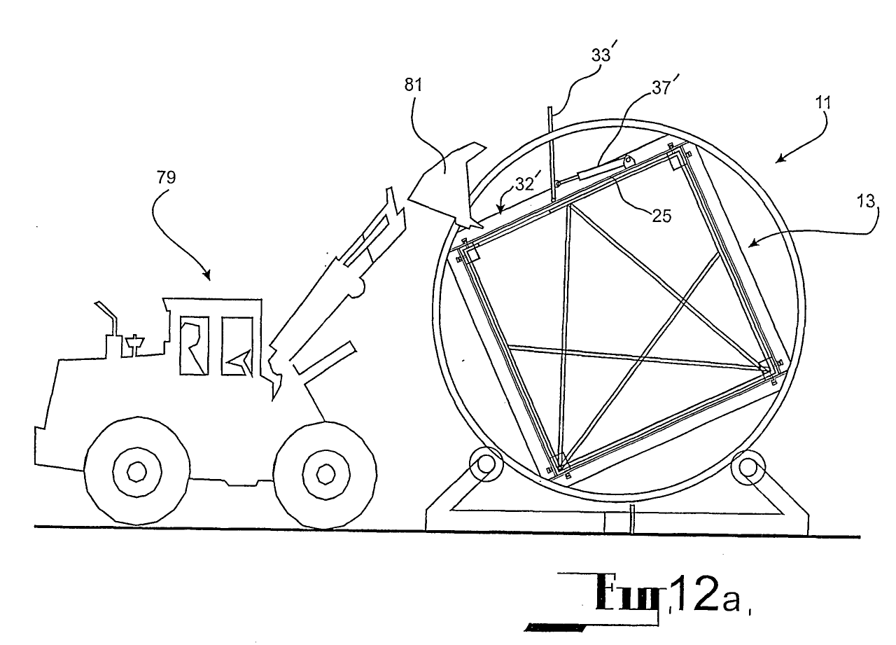

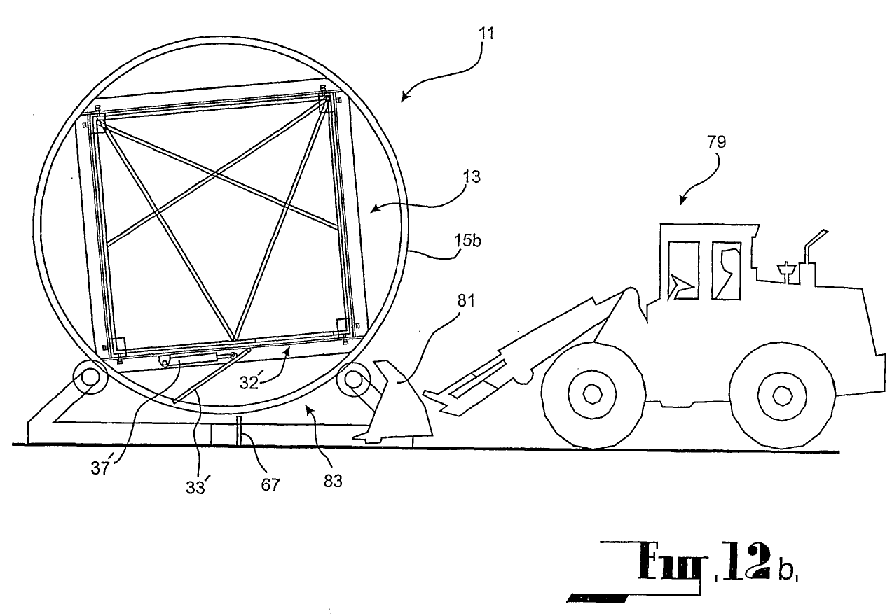

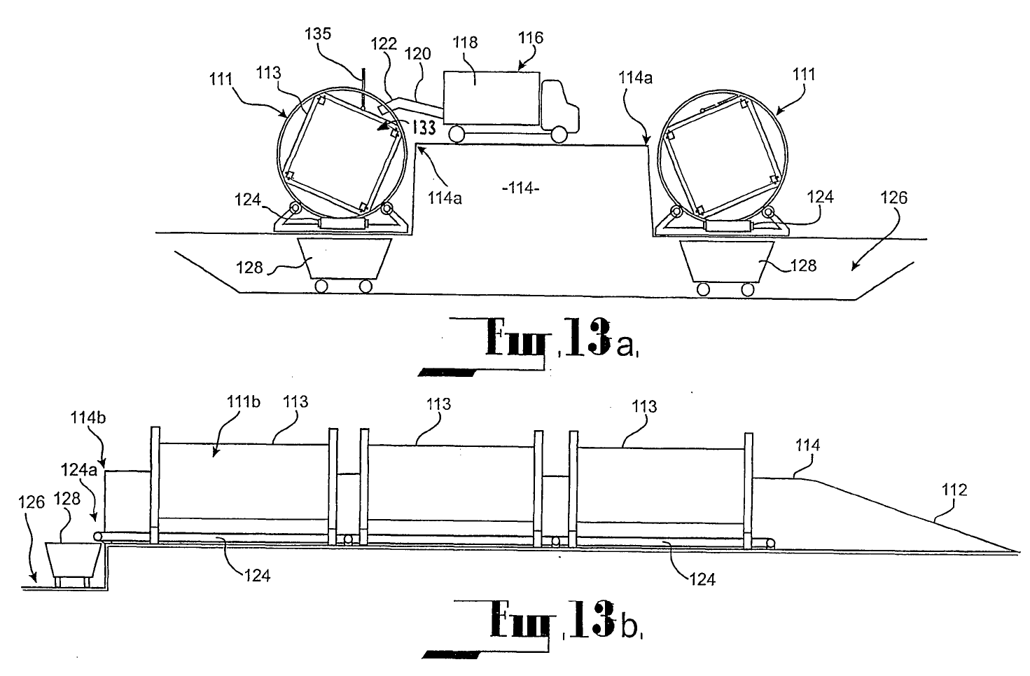

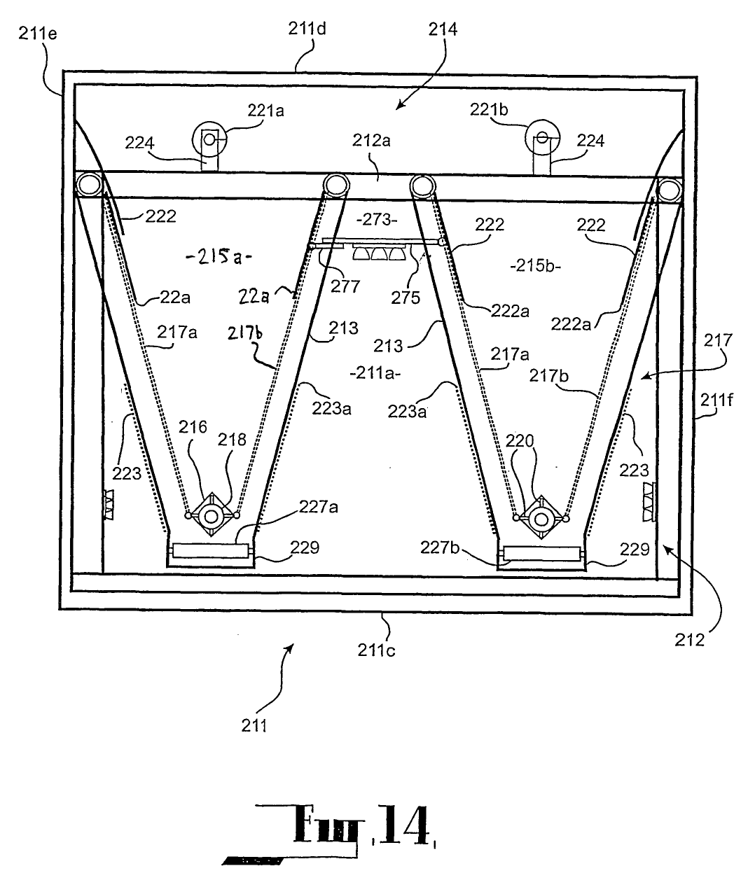

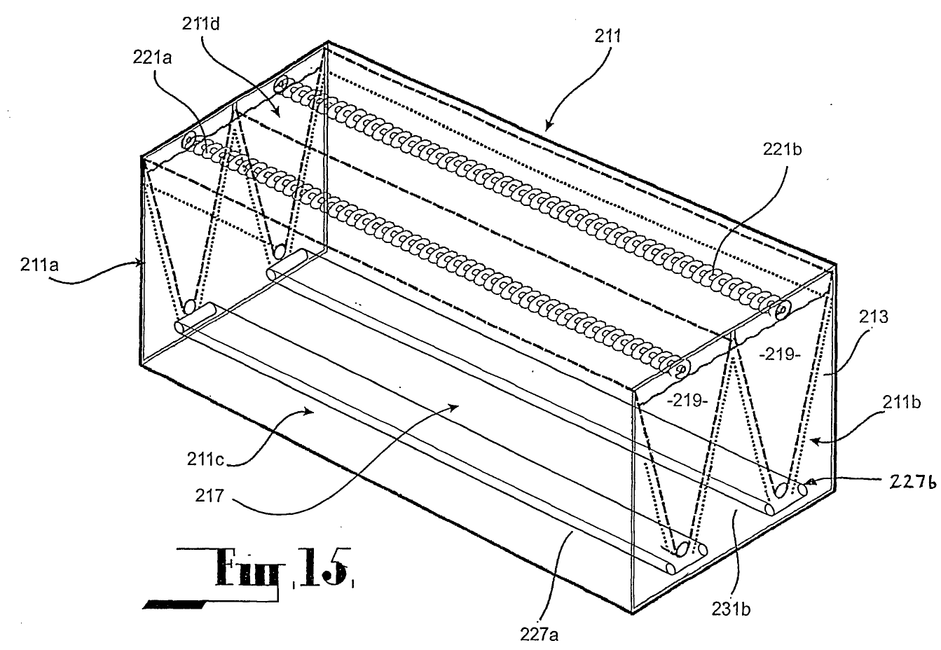

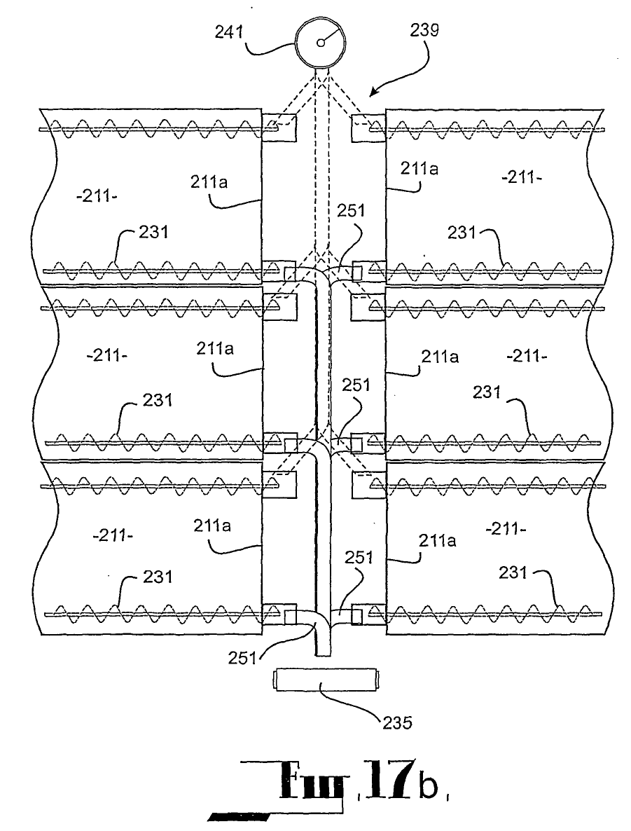

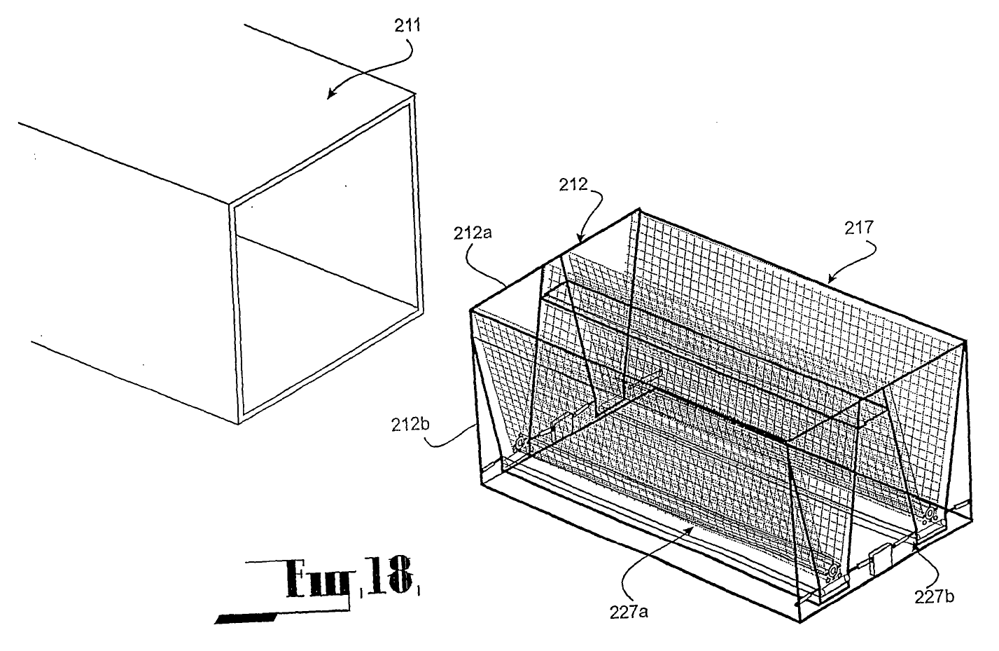

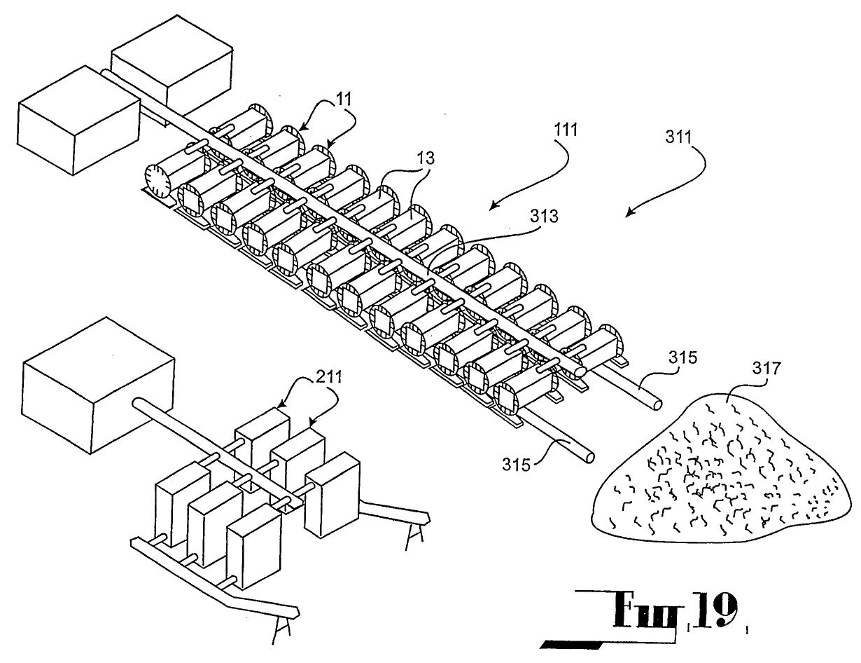

The presently preferred embodiment of the invention includes an apparatus for decomposing organic matter as described below in relation to FIGS. 1 to 8 of the drawings, in a common or related facility with a worm container arrangement as described below in relation to FIGS. 14 to 19 of the drawings. It will be appreciated that the apparatus for decomposing and the worm container arrangement described below are arranged in the common facility in any convenient inter-relationship which facilitates efficiency in transfer of the decomposed organic matter to the worm container arrangement. Any convenient form of transferring arrangement may be used, including those illustrated in FIGS. 12b and 13 of the drawings or an arrangement (not shown) in which the apparatus of FIGS. 1 to 8 discharges directly into the container(s) of FIGS. 14 to 18.

As shown in FIGS. 1 to 8 of the drawings, the apparatus 11 generally comprises a container module 13, a pair of wheel assemblies 15a and 15b, a framework 17, and a drive means 19.

The container module 13 used in the present embodiment is rectangular in cross-section and is in the form of a standard shipping container or seatainer having the standard internal dimensions of 12 meters long×2.33 metres wide×2.33 metres high. The container module 13 is of modular design, having standard interlocking means at each of the corners to enable it to be detachably mounted at either end to the wheel assemblies 15a and 15b, in a manner to be described in more detail later, and thus form an integral unit therewith.

The standard container module 13 comprises a floor 21, which is normally reinforced, two side walls 23a and 23b and a roof 25, which are normally not reinforced compared with the floor, a closed end 27 and an accessible end 29, normally provided with a pair of doors (not shown).

In its application in the present embodiment, the sides 23a, 23b and the roof 25 of the container module 13 need to be reinforced. Thus the side walls and the top of the shipping container are externally reinforced by a plurality of reinforcing members 31 configured in a truss arrangement fixedly juxtaposed thereto. The reinforcing members are rectangular hollow section (RHS) steel members and are welded directly to the sides and and top of the shipping container.

In order to inhibit or mitigate corrosion that arises from the composting process, the interior of the container module 13 is lined with welded polyethylene sheeting 32, bolted or adhered to all sides of the container interior. Fibreglass or polyurethane sheeting may be used in other embodiments to polyethylene, if more convenient to use.

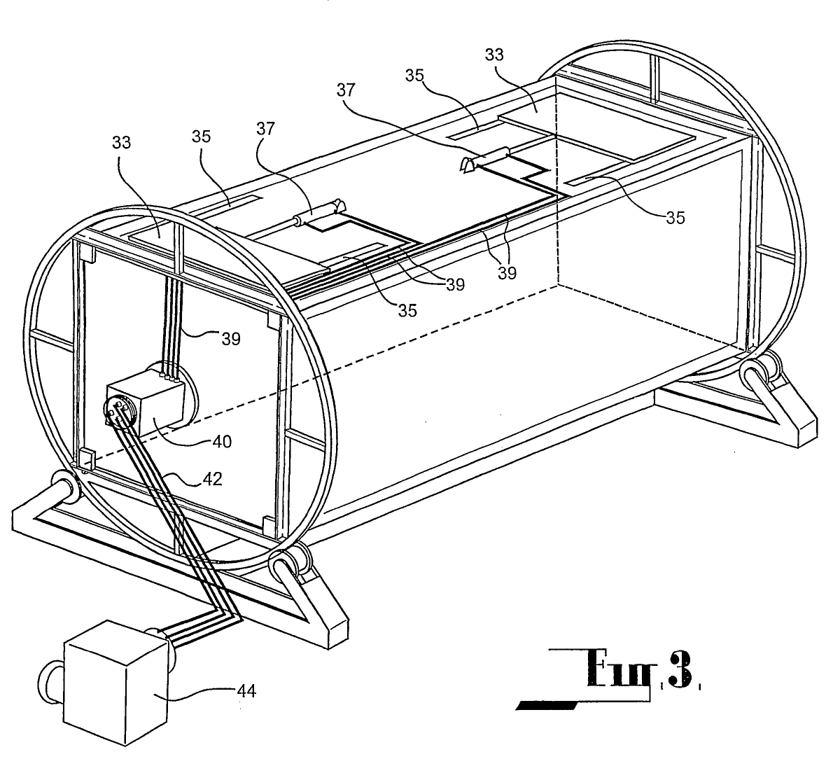

The roof 25 of the container module 13 is provided with an opening and a sealable closure 33 at either end thereof for filling and emptying the contents of the container. As shown in FIG. 3, the openings are closed by sliding the corresponding closures 33 along rails 35 extending axially along the container using corresponding hydraulic rams 37. The rams 37 are connected to hydraulic hoses 39 that are connected to a universal rotatable hydraulic coupling 40 disposed at one end of the container about a central bearing. The coupling in turn is connected via external hydraulic hoses 42 to a hydraulic power pack 44.

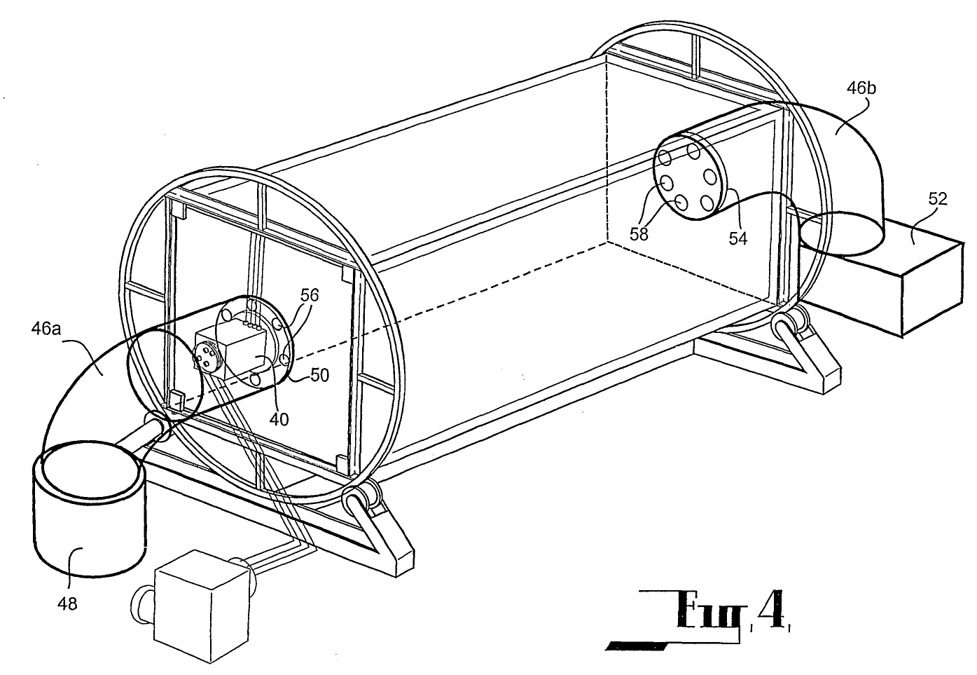

Standard seatainers are provided with fluid transfer valves towards the top of each side wall 23a and 23b. These valves are permanently closed where necessary to seal the container from the external atmosphere. In order to discharge the thermal energy generated by the composting process an air ventilation system is used that is shown in FIG. 4. The ventilation system comprises an inlet air hose 46a connected at one end to an external pressure vessel 48 and at the other end to an inlet universal rotatable air coupling 50, concentrically disposed and surmounting the hydraulic coupling 40, and an outlet air hose 46b connected at one end to an external filter vessel 52 and at the other end to an outlet universal rotatable air coupling 54. The pressure vessel 48 may house a compressor or a fan from which air is supplied to the container interior via the air hose 46 and apertures 56 slightly above atmospheric pressure. In this arrangement, the air within the container is vented through apertures 58 provided in the outlet air coupling 54 via the outlet hose 46b at the opposite end of the container by virtue of the positive pressure differential created between the interior of the container and the atmosphere. Alternatively, the external filter vessel 52 may comprise an extraction fan to create a negative pressure differential to extract air from the interior of the container.

The filter vessel 52 is of any convenient air filtering mass, such as carbon or organic or synthetic fibrous material.

The wheel assemblies 15a and 15b each comprise a rectangular frame 41 defining an inner accessway 43, fixing means 45 to releasably attach the frame to the container, and an outer circular rim 47.

The frame 41 is formed of right angle sections with outer webs 41a projecting radially outwardly from the central axis of the accessway 43 and inner webs 41b projecting axially of the central axis to define the sides of the accessway. The accessway 43 is commensurate in shape and marginally larger in size to circumscribe and accommodate one end of the container module 13 axially therein.,

The fixing means 45 comprises a stub plate 49 welded into each corner of the accessway 43 to the right angle sections of the frame 41, and a plurality of setscrews 51 and nuts (not shown). The nuts are welded into the slots provided on the interlocking means at each of the corners of the container module 13 at axial locations to receive axially disposed setscrews 51a mounted in the stub plates 49. These axial disposed nuts and screws 51a, fix the frame axially to the end of the container module 13 and allow fine adjustment of the frame 41 and hence the wheel assembly thereof, in the vertical plane. Transversely disposed setscrews 51b are threaded within the inner webs 41b, adjacent each corner to engage the interlocking means and fix the container laterally within the accessway 43 of the frame 41. Oppositely disposed transverse setscrews 51b essentially compress the sides of the interlocking means at each corner of the container module 13 together to fix the same to relative to the wheel assembly. In this manner minor variations in size between different sources of manufacture of seatainers are accommodated, notwithstanding that all seatainers are meant to be of a standard size.

The outer rim 47 is fixedly attached to the frame 41 directly at each of the corners of the frame and by radial bars 53 at intermediate locations of the frame. The rim 47 in the present embodiment is formed of rectangular hollow sections (RHS) curved to form a complete circle around the frame 41 and coaxial with the central axis of the wheel assembly.

The wheel assemblies are mounted to opposing ends of the container module 13. One wheel assembly 15a attached to the closed end 27 of the container is modified for direct connection to the drive means 19, in a manner to be described in more detail later.

The framework 17 essentially comprises a pair of support members 55a and 55b and an axially disposed cross member 57 tying the support members together.

The framework 17 supports the respective wheel assemblies and the container module as an integral unit in an elevated position and enables rotation of the same about the central axis thereof.

Each support member comprises a transversely disposed cross member 59, a pair of trunnions 61, each having a sheaved roller 63 mounted therein, and a pair of longitudinally disposed angle members fixedly mounted to each end of the cross member 59.

The trunnions 61 are disposed at each end of the cross member 59 and are angled radially inwardly so that the rollers 63 are disposed laterally either side of the axial cross member 57. The rotatable axis of each roller 63 is disposed parallel with the longitudinal extent of the framework 17 and the sheaves are aligned to form a bed within which the rim 47 of the corresponding wheel assembly may be seated vertically.

The angle members facilitate disposing the support members in an upright position, prior to and after mounting of the axial cross member 57. Accordingly, the axial cross member 57 is of a length commensurate to the length of the container module 13 so that the rollers 63 on opposing support members are spaced exactly the same distance apart as the rims 47of the wheel assemblies 15a and 15b, when mounted to the container module 13.

As shown in the drawings, the wheel assemblies 15a, 15b and the container module 13 as an integral unit are supported at an elevated position to the ground upon the framework 17. Moreover, the outer rims 47 of each wheel assembly are seated within the beds of the sheaved rollers 63 at either end of the framework 17 and are able to rotate as a unit therein.

In order to ensure proper seating of the opposing rims 47 within the rollers 63 to facilitate rotation of the wheel assemblies 15a, 15b and container module 13 about the central axis, the plane of each rim 47 needs to be precisely vertical and orthogonal to the central axis. Adjustment of the axial setscrews 51a enables this to be achieved relatively easily.

A kickboard 67 is vertically mounted to the axial cross member 57 to define a space on one side of the cross member 57 directly beneath the container to facilitate discharging and removal of the contents of a container.

The drive means 19 comprises one part connected to the support member 55a of the framework 17 and another part attached to the wheel assembly 15a. The drive means is a reciprocating clamp and release mechanism pivotally mounted to said framework and disposed tangentially to said wheel assembly in clamping and release engagement therewith, whereby said reciprocating mechanism includes control means to clampingly engage said wheel assembly during the extension stroke of said reciprocating mechanism, thereby imparting rotation to said wheel assembly from a reference position relative to said framework, and release said wheel assembly during the retraction stroke of said reciprocating mechanism to return said reciprocating mechanism to said reference position. Thus motion and power is transmitted from the reciprocating mechanism to the wheel assembly 15a to rotate the unit formed by wheel assemblies and the container module in conjunction with the rollers 63 about the central axis and relative to the framework 17.

The reciprocating mechanism is provided by a pair of cylinders positioned adjacent to the wheel assemblies, one cylinder to wheel assembly. Each cylinder has caliper means for releasably gripping the wheel assembly so that the cylinder pushes or pulls the assembly to partly rotate the framework and supported container, each cylinder gripping and pushing or pulling the assembly in turn to thereby rotate the assembly in the required manner. Thus the reciprocating mechanisms are controlled to operate in cooperation but out of phase with each other, so that whilst one reciprocating mechanism is clampingly engaged to said wheel assembly during said extension stroke thereof, the other reciprocating mechanism is released during said retraction stroke thereof, and vice versa.

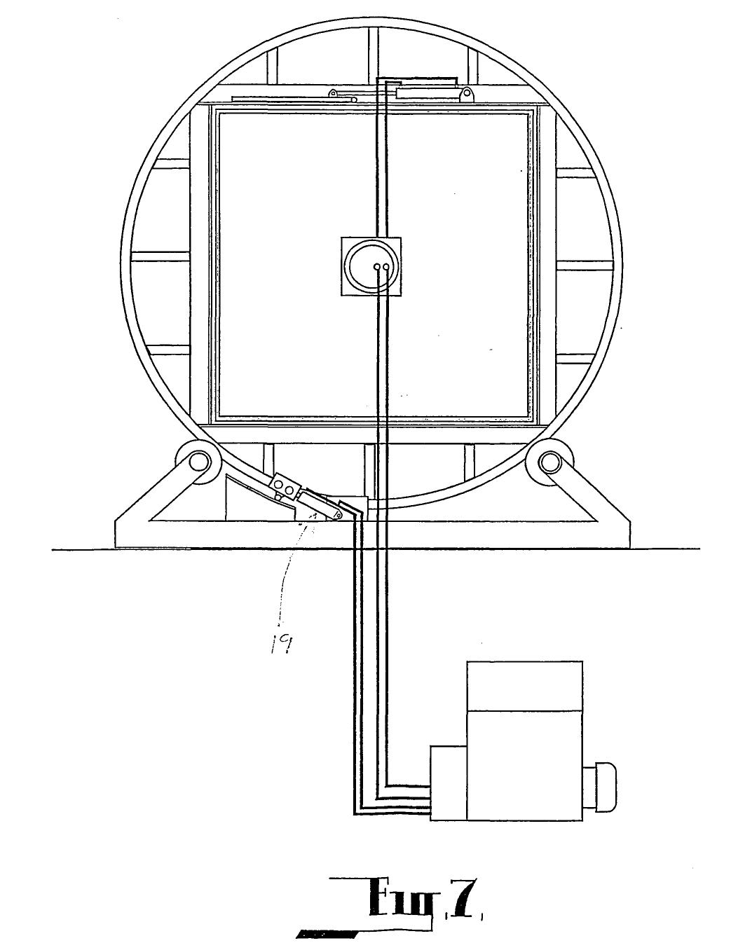

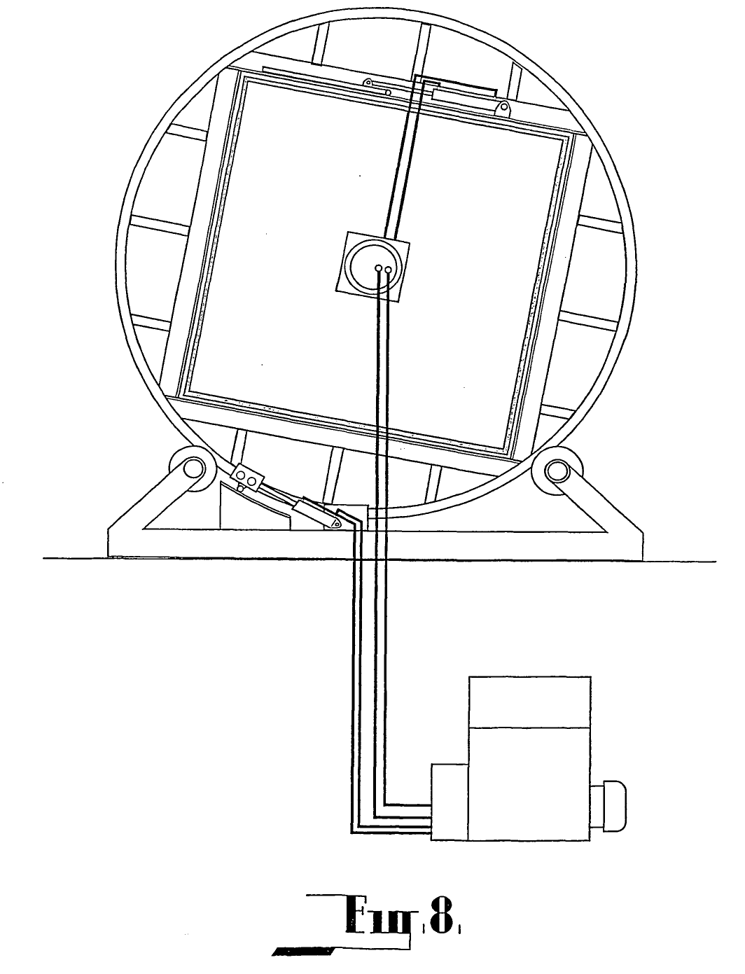

FIGS. 7 and 8 are end views showing the extension and retraction strokes of the reciprocating mechanism imparting rotational drive to the wheel assembly.

In an alternative embodiment a fixed clamp and release mechanism is fixedly mounted to said framework and disposed tangentially to said wheel assembly in clamping and release engagement therewith to work in cooperation with said reciprocating clamp and release mechanism, whereby said fixed mechanism includes control means to clampingly engage said wheel assembly during the retraction stroke of said reciprocating mechanism, thereby locking movement of said wheel assembly during said retraction stroke, and release said wheel assembly during the extension stroke of said reciprocating mechanism to allow rotation of said wheel assembly from said reference position.

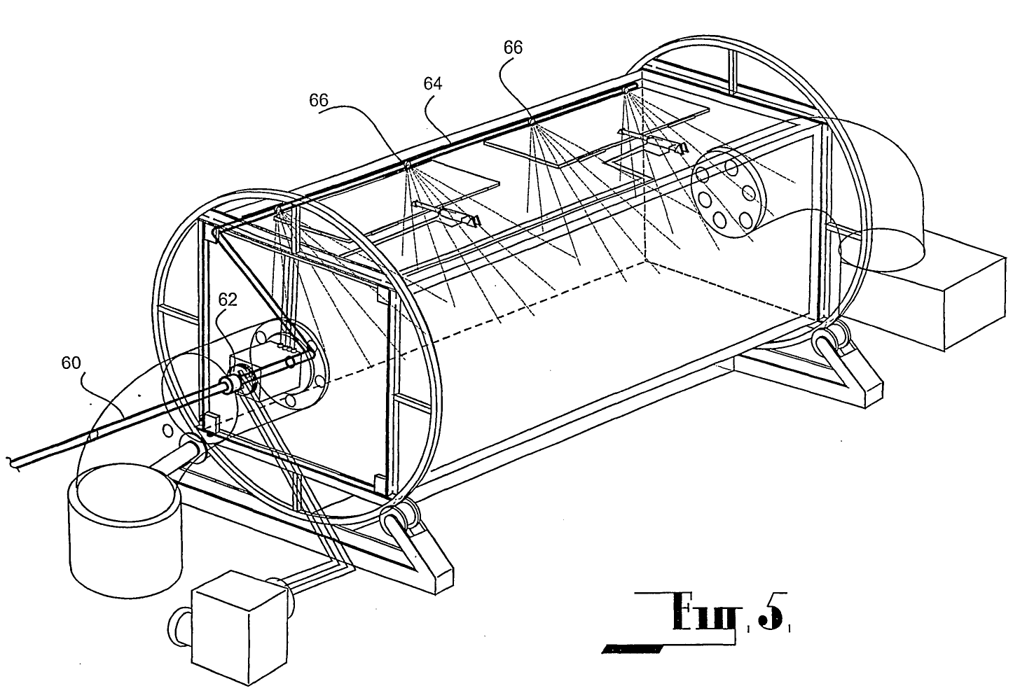

Moisture is introduced into the container in the manner as shown in FIG. 5 of the drawings, using a water pipe 60 and a water sealable universal coupling 62. A spray pipe 64 with nozzles 66 for spraying water into the interior of the container is provided.

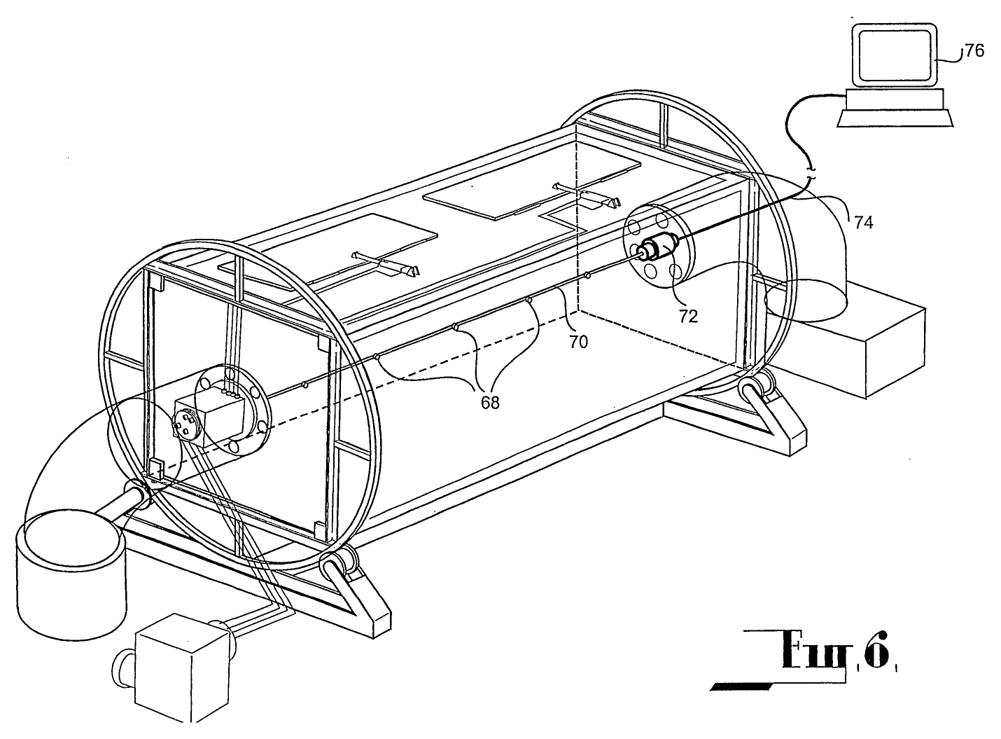

Introduction of air and moisture is controlled via a compurterised monitoring system, including various moisture, temperature, pH, carbon dioxide and oxygen sensors 68 mounted axially along a central standard 70 disposed within the container. These sensors are electically connected via an appropriate rotatable electrical coupling 72 and cable 74 to a computer terminal 76 for continuous monitoring as shown in FIG. 6.

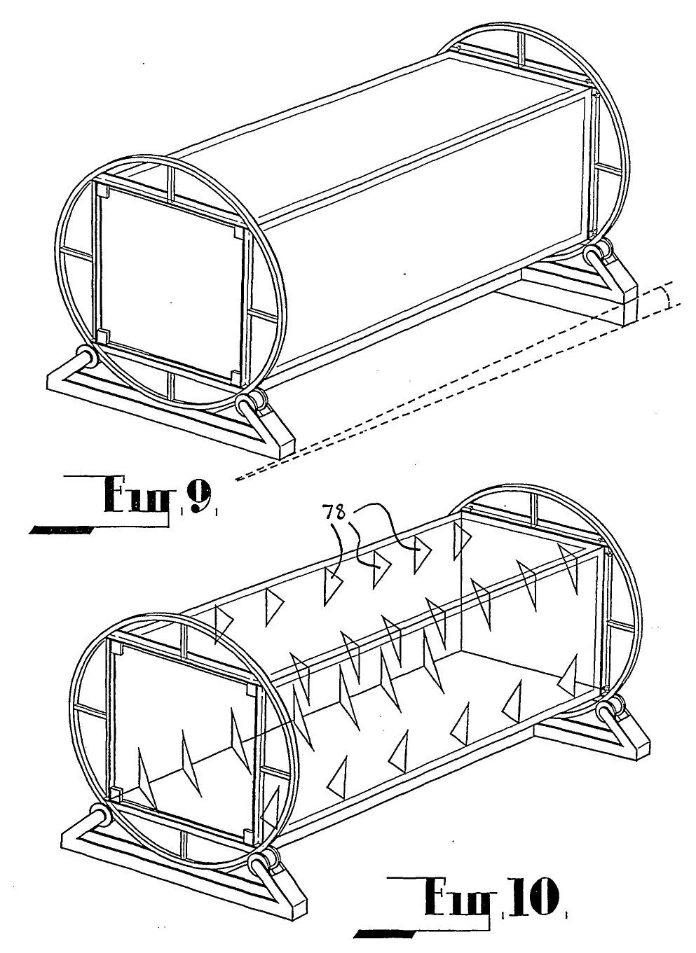

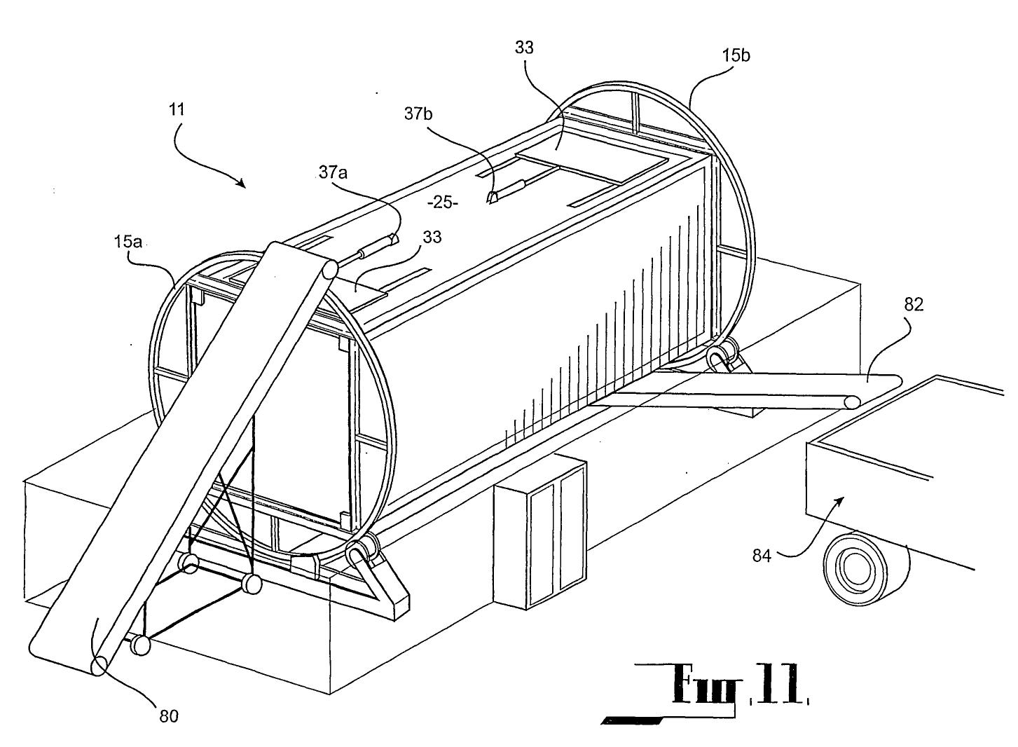

FIGS. 9 and 10 show two alternative ways of moving the contents of the container from one end of the container axially to the opposite end. This can be done by way of disposing the container on a slight downward gradient, for example 5°, from the loading end to the discharge end as shown in FIG. 9, or by the provision of angled paddles 78 as shown in FIG. 10.

The loading and discharging of the container is shown in FIG. 11, whereby an elevated loading conveyor 80 is provided to convey organic matter to be decomposed by the apparatus 11, over the wheel assembly 15a and into the opening provided at the in-feed end of the container when disposed in the position with the roof 25 at the top and the closure 33 retracted by the ram 37a. A depressed discharge conveyor 82 is disposed at the opposing end underneath the container to receive decomposed matter discharged from the container when the opening provided at the discharge end of the container is disposed in the inverted position with the roof 25 at the bottom and the closure 33 retracted by the ram 37b. In this position, the contents at the discharge end will fall under gravity onto the discharge container for conveying to a truck 84 or carriage for transport away from the apparatus.

In an alternative embodiment having openings and closures fitted longitudinally along the top and to one side of the container, as shown in FIGS. 12a and 12b, the container module 13 is initially filled by rotating it to the position shown in FIG. 12a, where the openings 32′ and closures 33′ are disposed in an uppermost position, angled slightly to the loading side of the apparatus. The rams 37′ are operated to open the closures 33′ to reveal the openings 32′, whereupon waste, containing organic residues in sufficient quantity for composting, is fed into the openings 33 by a front-end loader 79. Accordingly, the bucket 81 of the loader 79 can simply be elevated carrying waste to a position directly above an opening 32′ and then tipped to fall into the container module.

After the container module 13 is filled with waste to the prescribed level, the rams 37′ are operated to close the closures 33′ and seal the openings 32′.

After filling the container module 13 in either loading and discharging configuration, the container unit is rotated by operating the drive means via a timer (not shown) two or three revolutions, once or twice a day. The speed of revolution of the container module 13 is variable, depending upon the degree of composting required, the control parameters applied as a result of the sensing of conditions with the container and the budgetted power consumption of the apparatus. A typical speed of revolution is one revolution per 4 minutes. This is all that is required in most situations, with setting the correct mix and conditions, to obtain good thermophilic compost after approximately five to seven days.

The quality of the compost depends very much on the blend of waste fed into the container module. Typically the best blend is approximately 40% manure (dewatered biosolids, generally in the form of septage and food waste) and 60% green waste (hay, shredded tree clippings and the like). Compost made using animal waste is generally of higher value than compost made using human waste. However, one is paid for taking sludge comprising human waste, whereas sludge comprising animal waste needs to be purchased.

After five or six days have elapsed to enable adequate decomposition and composting of the waste within the container module 13, the container module is rotated to the inverted position with closures and openings disposed in a depressed position, which in the case of the alternative embodiment is as shown in FIG. 12b. In this embodiment, the openings 32′ and closures 33′ are disposed at their lowermost position, but slightly angled towards the discharge side of the apparatus. At this position, the rams 37′ are operated to open the closures 33′ and deposit the compost into the space 83 provided adjacent the kickboard 67 through the openings 31′. Accordingly, the front-end loader 79 can be operated to transfer compost from the space 83, using the kickboard 67 as a backboard for the bucket 81 of the loader to strike.

It should be noted that the container module 13 is supported at a sufficiently elevated position with respect to the ground to allow the bucket to access the space 83 and strike the kickboard 67 as appropriate to enable all compost deposited within the space to be removed.

Although not shown in the drawings, depending upon the temperature of the surrounding environment, the container module may be fitted with false walls along the side walls 23a, 23b and the roof 25. The false walls may be spaced approximately 20 to 30 centimetres from the actual side walls and roof of the container module 13 and be connected thereto by stanchions attached to the edges of the container. The false walls may comprise shade cloth or sheet metal, the latter being able to double as advertising hoardings, to provide an insulative space between the false wall and the actual walls and roof of the container. In this manner, the walls and roof are shielded from impinging sunlight which would otherwise heat the container to levels that would mitigate and even prevent the decomposition process.

In the case of using standard shipping containers, the floor is already insulated and hence does not require the provision of a false wall.

The use of the false walls as advertising hoardings enables the apparatus to have utility in locations that may be viewed by the public, i.e. adjacent roadways and thus increase the revenue stream that may be able to be generated from their use.

An important advantage of the present embodiment is that by using standard shipping container modules, the waste contained within the container is substantially sealed to prevent malodour during the composting process and thus reduce the incidence of air pollution and the transformation of malodorous and worthless waste to comparatively odourless and valuable compost.

In an alternative embodiment, the entire apparatus 11 may be disposed within a framework having false walls and roof to provide shading from the sun and hence an insulative effect to all parts of the container. These false walls may be formed with hinged closures to facilitate access to the main openings and closures 33 of the container module to facilitate filling and emptying of the same.

The second specific embodiment of the invention is directed towards the use of a multitude of apparatuses of the type described in the first embodiment to form part of a large scale system for decomposing large volumes of waste material on a scale that would be able to cater for the waste disposal needs of an urban sized community.

In this arrangement, as shown in FIGS. 13a and 13b, a series of apparatus 111 are disposed in two arrays so that a plurality of apparatus in each array, in this case three, are disposed adjacent to one another in an end-to-end configuration and are arranged to share a common filling and discharge facility.

In the present embodiment, the common filling facility comprises a ramp 112 and an elevated platform 114. Each array of apparatus 111 is disposed adjacent either side of the platform 114. The side edges 114a at either side of the platform are respectively disposed adjacent to the sides of the container modules 113 leaving a small marginal space between the outer extremities of the rims of the wheel assemblies of each apparatus 111 and the platform 114 for the wheel assembly and container unit to rotate. The height of the platform 114 is at a level generally horizontal with the openings 133 of the container modules when the container module is disposed with its closures 135 in an open position and the openings are at their uppermost juxtaposition to the platform edge 114a.

In this matter, a vehicle 116 fitted with a hopper bin 118 and auger discharge 120 may be driven up the ramp 112 full of waste and positioned adjacent to the platform edge 11a juxtaposed to a container module 113 that needs to be filled. The auger discharge 120 is then positioned so that its outlet chute 122 is disposed directly above the opening 133 of the container module to be filled. At this point, the auger can simply be operated and the contents of the hopper bin 118 fed into the container module 113.

The common discharge facility comprises a wide belt conveyor 124 disposed beneath each line of apparatus 111 on either side of the platform 114. The conveyor extends from the front apparatus 11a proximate to the ramp end of the platform 114 to the rear of the last apparatus 11b disposed at the opposite end 114b of the platform and sits directly beneath the lower most point of the container modules 113. Thus, the conveyor 124 extends longitudinally in parallel relationship to the central axis of the apparatus 111 to receive the discharge from a container module 113 when it is disposed at the emptying position with its closures 135 opened. In this arrangement, the kickboard is not required and is omitted to accommodate the conveyor.

At the rear end 114b of the platform, a pit 126 is provided within which the compost may simply be piled from the conveyor ends 124a, or, as shown in the drawings, skips 128 or trolleys may be located in the pit directly beneath the conveyor ends 124a to be loaded directly. When a skip is full, it is then transported to a remote location from where it may be more convenient to dispose of the compost.

An important feature of both the embodiments is the modularity of the system and the apparatus, which is provided by the use of standard size containers and most preferably shipping containers or seatainers. Although these containers come in different lengths, the apparatus can be used to receive and use either.

It should be appreciated that the present invention is not limited to the specific embodiments described herein. For example, whilst the embodiment describes two particular arrangements for in-feeding waste matter to the container module(s) and discharging decomposed composted matter therefrom, other differently engineered arrangements may be adopted, which do not depart from the spirit or scope of the present invention. In addition, although a reciprocating clamp and release arrangement has been described for the drive means, other drive means arrangements may be adopted, for example the chain and sprocket drive and the intermeshing gear drive may also be used instead, and consequently are deemed to fall within the scope of the present invention.

The decomposed material or compost produced as described above may be transported away directly after composting or alternatively may be used as the source organic matter for further, high level decomposing by the container 211 of FIGS. 14 to 18 described further below.

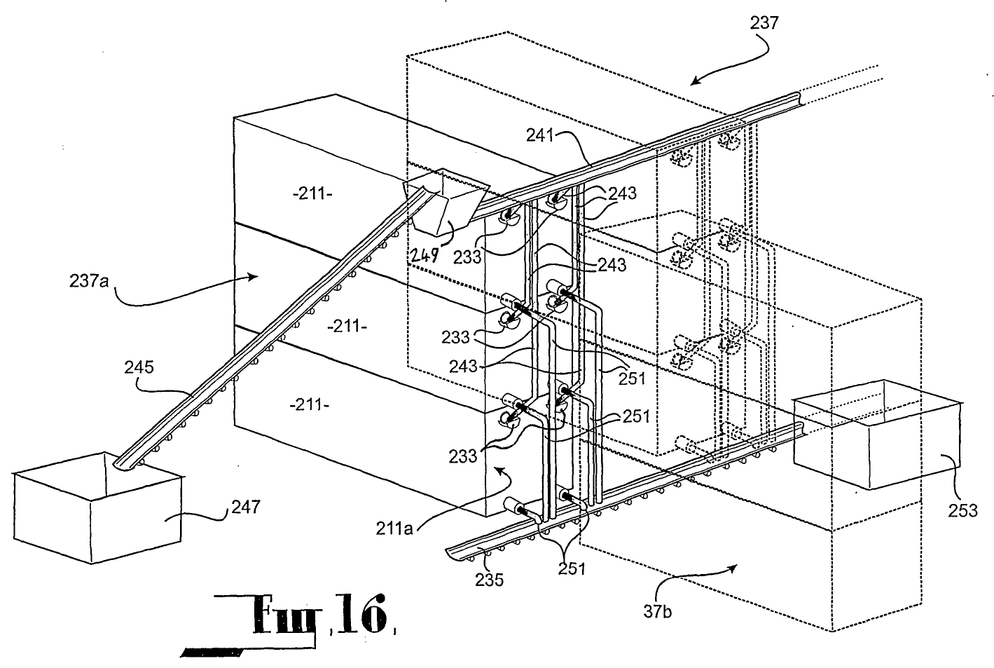

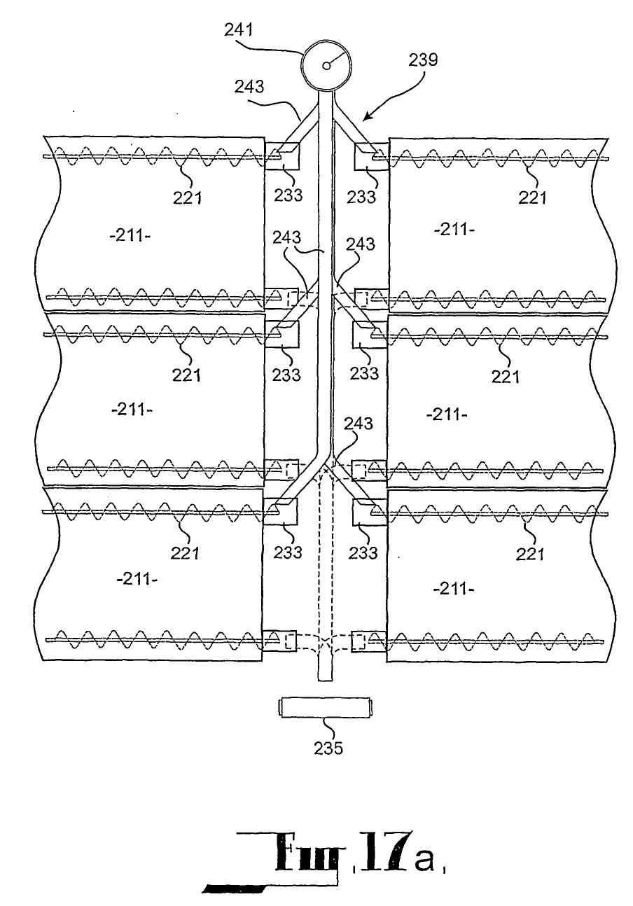

As shown in FIGS. 14 and 15, a container 11 is used which is in the form of a standard shipping container or seatainer having the standard internal dimensions of 12 metres long×2.33 metres wide×2.33 metres high. The containers are of modular design, each being particularly adapted with interlocking means to enable them to be stacked one on top of each other and alongside each other so as to form a rectangular array of rows and columns as shown in FIG. 16 of the drawings.

Each container 211 is closed at one end 211a and is provided with a closure in the form of a door 213 at the other end 211b to enable access into the container. A floor 211c, a roof 211d and two sides 211e and 211f also bound each container. The container 211 has mounted therein a sub-frame 212 comprising a cross member 212a and supporting legs 212b. The sub-frame 212 supports an inner housing 217 comprising two discrete, v-shaped compartments 215a and 215b for containing the organic matter within the container. In cross-section, as shown in FIG. 14, the v-shaped compartments 215 actually form corresponding isosceles triangles with the cross member 212a of the sub-frame forming the base of each triangle. The cross member 212a is spaced from the inside of the roof 211d to define a ceiling space 214 via which organic material may be fed into the container and into the compartments 215. The housing 217 has a pair of walls 217a and 217b for each compartment, which depend from the cross member 212a of the sub-frame.

The walls 217a and 217b constitute the equal sides of the triangle formed by each compartment and are formed of lengths of rigid mesh. In the present embodiment, the mesh comprises 50 mm×50 mm×5 mm arc mesh but may incorporate other dimensions to suit various forms of organic waste. The lengths of mesh are arranged in corresponding pairs to extend longitudinally from the one end 211a of the container to the other end 211b, and converge from the top to the bottom. Each pair of walls 217a and 217b formed of mesh is fixedly spaced apart by an auxiliary frame 216 at their base to accommodate a rotary valve 218 that closes the bottom of each compartment and forms the apex of the two v-shaped compartments 215. The auxiliary frame 216 is attached to the bottom edges of the adjacent walls by hinges.

The rotary valve 218 comprises four radial vanes 220 that define quadrants for dispensing organic matter from the base of the compartments 215 when rotated. The rotary valve has bearings and attaching pivot points every 1.5 to 3 metres. The rotary valve may be operated by an eccentric drive to generate some degree of vibration to facilitate the gravitational fall of decomposed material thereon and thus discharging of the same from within the confines of the compartments. Alternatively, or additionally, a small vibrating ram may be used to shake the mesh walls and help the gravitational fall of decomposed matter from the compartment.

The opposing axial ends of the v-shaped compartments 215a and 215b are closed with end panels 219 formed of the same type of mesh as the walls 217a and 217b to maximise the surface area of the compartments. Accordingly, the end panels 219a at the one end 211a are spaced therefrom and the panels 219b at the other end 211b are spaced from the door 213 when closed.

A marginal section of shade cloth 222 depends from the top of each side wall 217a and 217b on the inside of the compartment to close the holes of the mesh and thus prevent the escape of decomposed organic matter and indeed worms therethrough. The top 0.5 to 0.75 metres of the compartments 215 is where most of the earth worms reside and deposit eggs. Accordingly it desirable to retain the earth worms within the confines of the compartments as much as possible, and the provision of this section of shade cloth 222 helps achieve this.

It should be noted that when the container is disposed in its operative position, the floor 211c and the roof 211d are substantially horizontal, and the opposing ends 211a and 211b and the sides 211e and 211f are substantially vertical. Consequently, the base of the triangle defined by the compartment is horizontal and the median of the triangle is vertical. Importantly, the median of each compartment is substantially longer than the base of each compartment. This is to mitigate the passage of liquid from organic matter held with the housing 217, through the walls 217a and 217b, and promote the collection of liquid at the bottom of the housing.

An in-feeding means for feeding organic matter, and/or preferably decomposed matter from the apparatus of FIGS. 1 to 13 from outside of the container at the one end 211a to inside the respective compartments 215a and 215b of the container is provided in the form of a pair of screw feeding augers 221a and 221b. The feed augers 221a and 221b are supported by a pair of upright stanchions 224 connected to the cross frame 212a to dispose the augers below the roof 21 Id and spaced above the top of the compartments. The augers are disposed at traversely spaced locations to extend longitudinally along the container. Each feed auger 221 is equidistantly disposed from the walls 217a and 217b of each compartment so that each feed auger feeds a corresponding compartment with organic matter progressively from one end 211a of the container to the other end 211b. In addition, the augers 221 are spaced sufficiently above each compartment to mound the organic matter above the top of the compartments into the ceiling space 214 at an angle of repose with respect to the augers.

The feed augers 221 project through a pair of correspondingly disposed apertures in the one end 211a of the container, at transversely spaced apart locations proximate to the roof 211d. The external portions of the feed augers 221 are respectively reposed at the base of a corresponding pair of feed hoppers 233, located outside and mounted to the one end 211a of the container, to transfer waste organic matter from the feed hoppers 222 to the inside of the container. The feed augers 221a and 2221b are openly disposed within their respective compartments 15a and 215b, proximate the roof 211d to facilitate the intake of organic matter deposited into a corresponding feed hopper 233 and discharge of the same into the corresponding compartment 215a or 215b.

The walls 217a and 217b of the housing form apertures which are sized to be sufficiently small to retain organic matter within each compartment and sufficiently large to allow the passage of worm castings and liquid expelled from the organic matter on decomposing of the same therethrough.

Collection means for the worm castings and the liquid is provided at the bottom of each compartment 215 by means of a belt conveyor 227 and a drain 229. The drain simply comprises a drip tray that captures any liquid discharged from the decomposing organic matter that can be flushed or drained therefrom from time to time. The drain 229 is suspended above the floor 211c to facilitate cleaning.

As shown in FIG. 14 of the drawings, each wall 217 is flanked by guiding means 223 disposed in planar spaced relationship thereto, externally of the compartments 215a and 215b. The guiding means 223 flank the entire longitudinal and transverse extent of the outer walls 217a and 217b and the bottom part of the internal walls. The guiding means 223 in the present embodiments are perforated to allow light to pass therethrough and impinge the outer surface of organic matter retained within the walls 217.

In the present embodiment, the guiding means 223 are formed of 70% mesh shade cloth. The shade cloth is mounted with sufficient tension in its flanking position between the floor 211c and the roof 211d of the container, to deflect castings forced though the apertures of the mesh of the walls, and which fall under gravity, obliquely of the compartments, towards the collection means disposed at the bottom of the guiding means where the shade cloth is attached to the drain 229.

The bottom portion of the guiding means 223 on the inner walls 217a and 217b extends upwardly from the drain 229 and terminates along a horizontal edge 223a vertically below the bottom edge 222a of the marginal shade cloth portion 222. The reason for this is that the shade cloth 222 prevents castings and liquid from falling through the apertures of the mesh along the top of the housing walls and thus the guiding means only needs to function to catch egressed castings and liquid from below the bottom edge 222a. In addition, the inside area between the two compartments 215 provides an inspection area for an operator of to view the progress of the decomposing process and maintain the container, without being obscured by the guiding means.

The inside area between the two compartments 215 also provides for a worm collection means disposed within the region 273 which comprises a hinged trap door 275 and releasable lock 277.

The trap door 275 is normally closed and hingedly mounted at one end adjacent to the side wall 217a of the compartment 215b. It is spaced below the cross member 212a and provides a region in which organic matter may be disposed for worms to access between the two compartments. The trap door 275 normally reposes in a substantially horizontal position, but on releasing the closure 277, may be lowered at an oblique angle to form a sluice, along which the contents disposed on the door may be discharged either into a collection vessel, or onto the floor of the container.

A worm inlet means 279 is provided by the upper portion of the side walls 217b and 217a with the shade cloth portions 222 removed or elevated, leaving the mesh exposed having apertures of a size that allow worms through but not necessarily castings. In the present embodiment discrete sheets of mesh having an aperture size of approximately 3 mm square is affixed to the outside of the top portion of each side wall 217b and 217a. This mesh is provided with an inner flap 281 to overlie the upper edge of each of the longitudinal sides of the trough 175 to facilitate the containment of feed material within the confines of the trough and the worm inlet means 279, when the trough is in the reposed position.

Accordingly, worms can be periodically harvested from the compartments 215 by filling the region 273 with feed material and removing or lifting the shade cloth sections 222. In time, the worms will migrate from the confines of the compartments, through the worm inlet means 279 and into the feed material on the trap door 275. After a sufficient number of worms are in the trough, and the feed material is consumed, the trap door 275 can be lowered so that the opposing end may be disposed within an appropriate collection vessel and the closure released allowing the contents on the trap door, together with the worms to slide into the vessel. The castings or remaining organic material can be separated out and the worms on-sold or relocated.

The particular mesh size of the shade cloth is chosen so as to filter light passing therethrough to an extent to retain worms within the confines of the compartment but to retain castings and other composed matter impinging the same, within the confines of the flanked spaces of the compartments.

Lighting means in the form of lights 225 are respectively mounted on opposing side walls of the inside of the container, and between the two compartments, below the cross member 212a of the sub-frame, underneath the trap door 275. The lights extend axially along the container in a continuous or regularly spaced manner. The lights 225 are disposed towards the bottom half of the container to deter the movement of worms through the apertures of the walls 217 and to retain them within the organic matter, the worms being repelled by light.

The discharge belt conveyors 227 are disposed directly beneath each rotary valve 218 to catch material discharged therefrom and extend laterally to the sides of the corresponding drain 229 to also catch material discharged through the mesh walls 217a and 217b. Thus, castings and decomposed matter deflected to the bottom of the guiding means 223, externally of the walls 217, collects upon the belt, and on operation of the conveyor, is transferred axially along the bottom of the particular compartment associated therewith, to the one end 211a of the container. The belt is pervious to liquid, allowing it to pass through and be collected in the drain 229.

The container 211 also includes a moisture and climate control system (not shown) incorporating a reverse cycle air conditioner with electronic thermostat controls and moisture probes which will be connected to an automated irrigation system—both to maintain ideal moisture and temperature suited to maximum breeding and waste consumption by compost worms.

As with the feed augers 221a and 221b, the conveyor belts 227a and 227b extend longitudinally along the container, through corresponding apertures provided in the end of 211a of the container, close to the floor 211c, to discharge collected castings and decomposed matter onto a main discharge conveyor 235. The main discharge conveyor 235 is disposed externally of the container adjacent to the end 211a of the container in the bottom row of the array, and will be described in more detail later.

The arrangement of the feed hoppers 233 and the main discharge conveyor 235 in the array of containers 211 is more particularly shown in FIG. 16 of the drawings. As can be seen from this figure, the containers 211 are stacked in a matrix 237 comprising a pair of arrays 237a and 237b, each array 237 comprising three horizontal rows in height and a plurality of vertical columns. The containers in each array are arranged so that the one ends 211a thereof are disposed in vertical alignment to define a common end of each array. Further, the common ends of each array 237 are oppositely disposed in confronting and spaced apart relationship to define a corridor 239 which extends orthogonally of the longitudinal axes of all the containers. The corridor 239 accommodates a main delivery auger 241 for delivering waste organic matter to all of the containers and the main discharge conveyor 235 for discharging castings and other composted material from each of the containers.

The main delivery auger 241 is disposed above the top row of containers of both arrays and extends along the corridor 239, intermediate the common ends of the arrays. A series of feed chutes 243 depend from the main delivery auger 241 and feed waste organic matter to each of the feed hoppers 233 under gravity, from the main delivery auger. Thus a feed chute is associated with each feed hopper. As previously described, the feed hoppers 233 of each container are arranged so that the outer extent of each of the feed augers 221a and 221b are disposed at the base of each hopper, respectively. Each feed chute 243 has an electronically operated gate, which is selectively operated to charge the feed hopper associated therewith, with waste organic matter for subsequent in-feeding into the corresponding compartment of the container by means of the particular feed auger.

The input side of the main delivery auger 241 is supplied with waste organic matter by a belt conveyor 245, which is connected to a main mixing hopper 247 at ground level. The mixing hopper 247 is a large shredder where waste organic material, and/or decomposed organic matter from the apparatus of FIGS. 1 to 13, can be deposited into it from a truck or other conveyance. The mixing hopper 247 will blend and shred the waste material into small particles which will then be conveyed by the belt conveyor 245 to a main feed hopper 249 to which the main delivery auger 241 is connected. This waste organic matter may be quire liquid to facilitate the environment for the worms in each container and thus lends itself to be gravity fed via the feed chutes 243 into each feed hopper 233 in sequence.

Microswitches (not shown) are associated with each container, feed hopper 233 and gate associated therewith, so that one microswitch may indicate when a compartment needs replenishing with organic matter and activate the appropriate feed auger associated with than compartment, another microswitch may indicate when the feed hopper 233 is empty and opens the gate of the particular feed chute associated with that feed hopper to release organic matter from the main delivery auger 241. The main delivery auger 241 may be triggered to operate in such a situation if it is not already doing so and stop in response to the absence of microswitch activation of a gate. A further microswitch may be associated with each feed hopper 233 to indicate when the particular hopper is full with organic matter and close the gate of the chute supplying the hopper before it overfills.

As can be seen, the feeding cycle for the containers works in a sequential manner, whereby as one container fills to capacity with organic matter, organic matter is conveyed to the next container, and so on, until all of the containers are filled. Consequently the main delivery auger 241 is virtually continuously operated to keep all of the containers filled with organic matter.

Essentially, all of the compartments 215 of each container are successively filled with organic matter from the one end 211a, serially along the longitudinal extent of the container, until the entire contents of the compartment are filled to capacity.

The compartments are primed with worms to feed and digest the organic matter so that worm castings are excreted, worm castings being essentially decomposed organic matter.

The majority of the worms operate near the outer surface of the compartments and hence this is where the most of the castings are excreted. Fresh organic matter near the centre of the compartments and on the upper surface is usually composting, conditions that the worms prefer to avoid. Consequently, the worms migrate to the outer surface of the compartments, adjacent to the walls 217, where there is more oxygen, moisture and ideal temperatures. The castings are dried by the reverse cycle air conditioner, whereupon they spill through the apertures of the walls 217 under the action of gravity. Consequently, they strike the deflector panels 219 and collect in the discharge augers 231.

The external portions of each of the discharge augers 231 for the upper rows of containers are arranged to direct discharged castings and decomposed matter from the bottom of the containers into discharge chutes 251 connected thereto. The discharge chutes 251 are disposed so as to direct the discharged matter into the main discharge conveyor 235. The main discharge conveyor 235 is disposed beneath the main delivery auger 241, intermediate the common ends of the arrays, along the corridor 239.

On operation of the discharge augers 231, discharged matter will be deposited onto the main discharge conveyor 235, and be conveyed therealong to the end of the corridor 239, opposite to the direction of the main delivery auger 241, for ultimate discharge into a stockpile contained within a shed 253 or other collection facility, remote from the containers.

Appropriate microswitch may be provided to sense filling of the discharge augers, before triggering operation of the main discharge conveyor 235. Alternatively, the discharge augers and the conveyor may simply be run continuously to discharge castings and decomposed matter as it is collected in the discharge augers 233.

A reticulated drainage system (not shown) is connected to each of the drains 229 to dispense liquid collected therein as it is collected. This system comprises a network of drainage pipes, which are connected via a suitable drain at each end of a trough proximate to the one end of each container. The drainage pipes extend through the wall at the one end of each container and work under gravity to dispense the liquid as it accumulates. Accordingly, the troughs are mounted with an appropriate grade to enable the accumulated liquid to run off through the drain at the end of the trough.

By virtue of this arrangement, a convenient setup for the plant is achieved with there being a single easily accessible mixing hopper at one end of the corridor between the confronting common ends of the arrays of containers for dropping off waste organic matter to be subsequently disseminated to each of the containers in an automated manner. Simultaneously, the plant provides for a single easily accessible stockpile of decomposed or composted matter at the opposite end of the corridor for automatically collecting castings and decomposed matter from each of the containers for subsequent utilisation. The stockpile may or may not be disposed within a shed or housing for sheltering from the weather.

The third embodiment of the present invention is substantially similar to the first embodiment except that the internal design of the container is marginally different. To facilitate understanding the description, the same sequence of reference numerals commencing from 100 have been used to identify corresponding components of the container.

In a further embodiment, the arrangements of the first and second embodiments incorporating a multitude of apparatuses 11 and containers 211 are combined as shown in FIG. 19 to provide a system 311. The system 311 comprises an array of apparatuses 111 that may be arranged in any convenient manner to receive organic matter from a common auger delivery system 313. The organic matter is decomposed within the container modules 13 in parallel with each other over time, and is subsequently output to a common conveyer system 315. The decomposed or partly decomposed material may then be stockpiled 317 and despatched for use elsewhere or subsequently input as the souce material to an array of containers 211 for higher order decomposing.

It should be appreciated that a number of advantages are provided by the present invention in relation to waste disposal on a large scale. Moreover, an important advantage is space, whereby an area of only 100 metres long and 30 metres wide could contain 210 containers with a common feed and discharge or harvesting system (35 containers long, 2 wide and 3 high). This area and 150 tonnes of worms would process approximately 100 tonnes of waste per day. Income could come from waste fees (eg landfill), surplus worms as they breed, and the sale of organic fertiliser.

It should be appreciated that the present invention is not limited to the specific embodiment described herein, and that variations and modification in accordance with common engineering knowledge in the design and construction of a system for treating organic matter using the same general principles and concepts described herein in whole or in part, would still fall within the scope of the present invention. For example, whilst the embodiment describes one particular arrangement for automatically in-feeding organic matter to the containers and automatically discharging castings and decomposed matter from the containers, other differently engineered arrangements may be adopted, which do not depart from the spirit or scope of the present invention.

Further still, the invention is not limited to the specific rectangular array of containers described in the embodiment. For example a circular array of containers may be envisaged, which have a central hub for location of the in-feeding and discharging infrastructure, whereby the containers are arranged in a radial manner around the hub.

Furthermore, the invention is not limited to the particular aspect of the casting agitating means and worm collection means described in the second embodiment and that other arrangements that may involve the same principles ares still not considered to fall within the scope of the invention.

The claims defining the invention are as follows:

1. A container for the treatment of organic matter involving the use of worms to decompose the organic matter and produce castings, the container including:
at least one inner housing for accommodating organic matter and worms within the confines of the container;
an in-feeding means for feeding organic matter from outside of said container at a first end, to inside and along the container, so as to fill and maintain the housing with a supply of organic matter for treatment by the worms;
the or each housing having V-shaped walls formed with apertures sufficiently small to retain the organic matter within he housing and sufficiently large to allow for the passage of worm castings and liquid expelled from said organic matter therethrough;
the or each housing being also provided with a rotary valve disposed in an apex of the or each housing, arranged to dispense treated organic matter therefrom; and
a belt conveyor disposed beneath the or each inner housing, arranged to selectively collect said worm castings and to discharge said worm castings from the container, or to selectively collect treated organic matter dispenses from the or each inner housing by the rotary valve and to discharge said treated matter from the container.

2. A container as claimed in claim 1, wherein a closure is disposed at a second end of the container for allowing access to said housing and said collection means.

3. A container as claimed in claim 1 or 2, wherein the in-feeding means is assisted with electronic micro-switch and computerised technology to keep the container at optimal levels of capacity.

4. A container as claimed in any one of the preceding claims, including lighting means to illuminate the outer surface of organic matter reposed within said housing.

5. A system for treating organic matter involving the use of works to decompose the organic matter and produce castings, comprising;
a plurality of containers as claimed in any one of claims 1 to 4,
a common organic matter delivery means linking all of said containers for feeding said in-feeding means of each container with organic matter; and
a common discharge means linking all of said containers for discharging castings and/or liquid from said discharge means of each container;
wherein said containers are rectangular and modular.

6. A system as claimed in claim 5, wherein said plurality of containers comprises:
an array of rectangular, stackable containers arranged into one or more regular vertical columns and one or more horizontal rows;
said containers being arranged in said array so that said one end of each said container is disposed at a common end of said array;
a main organic matter feed means for supplying organic matter from a main supply area externally of said array to said common organic matter delivery means and thence to said in-feeding means of each said container at said common end of the array; and
said common discharge means also be disposed at said common end of the array for conveying castings and/or liquid from said discharge means of each said container from said common end to a main discharge area externally of said array.

7. A system as claimed in claim 5 or 6, wherein said containers are arranged in said array so that the longitudinal axis of each container is disposed in parallel spaced relation to each other container, and the one ends of the containers are disposed in rectilinear alignment to define said common end.

8. An apparatus for decomposing organic matter involving the use of a replaceable container module comprising:
a pair of wheel assemblies for detachably mounting to opposing ends of a container module to form an integral unit when fixedly connected thereto;
a framework for supporting the wheel assemblies in an elevated position when the container module is supported therebetween as an integral unit for rotation about a central longitudinal axis; and
drive means associated with at least one wheel assembly to drive continuous rotation of said one wheel assembly relative to the framework about the central longitudinal axis, whereby the interconnectivity of the wheel assemblies to the container module permits the container module to be rotated continuously as a whole.

9. An apparatus as claimed in claim 8, wherein each wheel assembly has:
(i) an inner accessway generally commensurate in shape to circumscribe and accommodate the end of a container module axially therein;
(ii) fixing means disposed within said accessway to releasably attach said wheel assembly thereto; and
(iii) an outer rim, rotatable about a central longitudinal axis of the wheel assembly.

10. An apparatus as claimed in claim 8 or 9, wherein said framework comprises a pair of end support members for rotatably supporting the respective wheel assemblies in an upright position.

11. An apparatus as claimed in claim 10, wherein each said support member has a pair of transversely spaced sheaved rollers to accommodate the rim of a corresponding wheel assembly therein in coplanar relationship therewith so that the wheel assembly reposes at the elevated position and is able to rotate about the central axis at this position in conjunction with the rollers.

12. An apparatus as claimed in any one of claims 8 to 11, wherein said container module is rectangular in cross-section and is provided with an opening and a sealable closure along one side thereof for filling and emptying of the contents thereof.

13. An apparatus as claimed in claim 12, wherein a said opening and sealable closure for filling said container module is disposed at one end of said one side,
and a said opening and sealable closure for emptying the contents of said container module is disposed at the opposing end of said one side.

14. An apparatus as claimed in any one of claims 8 to 13, wherein said drive means comprises a reciprocating clamp and release mechanism pivotally mounted to said framework and disposed tangentially to said wheel assembly in clamping and release engagement therewith, whereby said reciprocating mechanism includes control means to clampingly engaged said wheel assembly during the extension strokes of said reciprocating mechanism, thereby imparting rotation to said wheel assembly from a reference position relative to said framework, and release said wheel assembly during the retraction stroke of said reciprocating mechanism to return said reciprocating mechanism to said reference position.

15. An apparatus as claimed in claim 14, wherein a fixed clamp and release mechanism is fixedly mounted to said framework and disposed tangentially to said wheel assembly in clamping and release engagement therewith to work in co-operation with said reciprocating clamp and release mechanism, whereby said fixed mechanism includes control means to clampingly engage said wheel assembly during the retraction stroke of said reciprocating mechanism, thereby locking movement of said wheel assembly during said retraction stroke, and release said wheel assembly during the extension stroke of said reciprocating mechanism to allow rotation of said wheel assembly from said reference position.

16. An apparatus as claimed in claim 14, wherein a pair of said reciprocating clamp and release mechanisms are mounted to said framework and wheel assembly and are controlled to operate in co-operation but out of phase with each other, so that whilst one reciprocating mechanism is clampingly engaged to said wheel assembly during said extension stroke thereof, the other reciprocating mechanism is released during said retraction stroke thereof, and vice versa.

17. An apparatus as claimed in any one of claims 8 to 16, wherein said container module comprises a standard shipping container.

18. An apparatus as claimed in claim 17, wherein said side walls and the top of the shipping container are externally reinforced by a plurality of reinforced members configured in a truss arrangement fixedly juxtaposed thereto.

19. An apparatus as claimed in any one of claims 8 to 18, wherein said framework is provided with a longitudinally disposed upright kick-board extending axially between the opposing ends of the framework to define a space either side thereof at the base of the apparatus for emptying the contents of the container module and facilitating removal therefrom.

20. An apparatus for decomposing organic matter comprising:
a pair of wheel assemblies for detachably mounting to opposing ends of a container module to form an integral unit when fixedly connected thereto;
a framework for supporting the wheel assemblies in an elevated position when the container module is supported therebetween as an integral unit for rotation about a central longitudinal axis; and
drive means associated with at least one wheel assembly to drive continuous rotation of said one wheel assembly relative to the framework about the central longitudinal axis, whereby the interconnectivity of the wheel assemblies to the container module permits the container module to be continously rotated as a whole;
wherein the unit can be rotated to a filling position at which the container module can be filled with material containing organic residues and to an emptying position at which the container module can be discharged of its contents.

21. A system for handling the disposal of large volumes of waste containing organic matter comprising:
a plurality of apparatuses as claimed in any one of claims 8 to 20, the plurality being arranged in an array so that two or more apparatuses in the array are disposed adjacent to one another to share a common filling or discharge facility.

22. A system as claimed in claim 21, wherein said common filling facility is a ramp and elevated platform disposed adjacent to a plurality of said apparatuses disposed serially in end-to-end relationship to each other, the edge of the
platform being disposed marginally adjacent to the sides of the container modules at a level generally horizontal with the opening of a container module when the container module is disposed with the closure in an open position and the opening is at its uppermost juxtaposition to the platform edge.

23. A system as claimed in claim 21 or 22, wherein said common discharge facility comprises a wide belt conveyor disposed beneath the lowermost point of the container modules, extending longitudinally in parallel relationship to the central axis of each apparatus.

24. A system as claimed in any one of claims 21 to 23, wherein decomposed organic matter discharged by said apparatuses is transferred to a container as claimed in any one of claims 1 to 4, or a system as claimed in any one of claims 5 to 7, wherein said decomposed organic matter becomes the source of organic matter outside of said container for said in-feeding means.
